(19) United States
(12) Patent Application Publication
(10) Pub. No.: US 2026/0130775 A1
(43) Pub. Date: May 14, 2026

(54) Gradated Silicone Shade Guide

(71) Applicant: The Government of The United States, as represented by The Director of the Defense Health Agency, Fort Detrick, MD (US)

(72) Inventors: Steven E. Handel, Fort Eisenhower, GA (US); James Pizzini, San Antonio, TX (US); Ethan Hart, Lakehills, TX (US)

(73) Assignee: The Government of The United States, as represented by The Director of the Defense Health Agency, Fort Detrick, MD (US)

(21) Appl. No.: 19/388,427

(22) Filed: Nov. 13, 2025

Related U.S. Application Data
(60) Provisional application No. 63/720,289, filed on Nov. 14, 2024.

(51) Int. Cl. A61F 2/50; A61F 2/76; G01J 3/46

(52) U.S. Cl. CPC A61F 2/5046; G01J 3/463; A61F 2002/5001; A61F 2002/7615

(57) ABSTRACT

A gradated shade guide and method for using the same for color matching during prosthesis manufacture. A gradated shade guide for visual assessment of shade of a tinted material compared to a patient's skin tone at a plurality of depths of the tinted material, the gradated shade carrier operable to house for inspection varying thicknesses of the tinted material corresponding to the plurality of depths. A method includes determining a thickness of the tinted material to achieve a color match of the tinted material compared to the patient's skin tone using a gradated shade guide that provides for visual assessment of shade of the tinted material, the gradated shade guide operable to house for inspection varying thicknesses of the tinted material corresponding to a plurality of depths and where said determining the thickness includes comparing to the patient's skin tone the tinted material housed at the plurality of depths in the gradated shade guide.

### PRIORITY CLAIM

This application claims the benefit of provisional application Ser. No. 63/720,289 filed Nov. 14, 2024 and titled “Gradated Silicone Shade Guide” the entire contents of which are hereby incorporated by reference.

### STATEMENT OF GOVERNMENT INTEREST

The invention described herein may be manufactured, used and licensed by or for the United States Government.

### BACKGROUND

Maxillofacial prosthetics is the branch of prosthodontics concerned with the restoration and replacement of stomatognathic and craniofacial structures with prostheses that may or may not be removed on a regular or elective basis. The prosthetic concealment of a congenital or acquired extraoral defect is challenging and heavily relies on the ability of the maxillofacial prosthodontist or anaplastologist to select the correct material, re-create the missing anatomy, and blend the correct tints to match the patient's skin tone. The re-creation of the missing anatomy relies upon the artistic ability of the individual fabricating the prosthesis and without the appropriate tone selection, a prosthesis will never be well-concealed. Color matching is typically completed by mixing pigments and rayon flocking into medical-grade silicone and using a flat carrier to assess the accuracy of the blend. This process does not account for how material thickness affects the appearance of the silicone.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings provide visual representations which will be used to describe various representative embodiments more fully and can be used by those skilled in the art to better understand the representative embodiments disclosed and their inherent advantages. In these drawings, like reference numerals identify corresponding or analogous elements.

FIG. 1A depicts an example of a traditional shade carrier while FIG. 1B illustrates a gradated shade guide, in accordance with various embodiments of the present disclosure.

FIG. 2 depicts a digital design of a gradated silicone shade guide, in accordance with various embodiments of the present disclosure.

FIG. 3 illustrates two variations of three-dimensional printed gradated silicone shade guides, in accordance with various embodiments of the present disclosure.

FIG. 4 illustrates shade matching of unexposed skin, in accordance with various embodiments of the present disclosure.

FIG. 5 illustrates variations of silicone tones ready to be dispensed during prosthesis fabrication, in accordance with various embodiments of the present disclosure.

FIG. 6 illustrates an example final prosthesis, in accordance with various embodiments of the present disclosure.

FIG. 7 illustrates delivery of an example final prosthesis on the patient, in accordance with various embodiments of the present disclosure.

FIG. 8 illustrates the example final prosthesis worn by the patient, in accordance with various embodiments of the present disclosure.

FIG. 9 illustrates gradated silicone shade guides with various colors of silicone, in accordance with various embodiments of the present disclosure.

FIG. 10 illustrates gradated silicone shade guides with grips or handles and filled with medical grade silicone, in accordance with various embodiments of the present disclosure.

FIG. 11 illustrates still further configurations of gradated silicone shade guides with grips or handles, in accordance with various embodiments of the present disclosure.

### DETAILED DESCRIPTION

Shade matching of extraoral prostheses is completed by mixing pigments and rayon flocking into medical-grade silicone and using a carrier to evaluate the color match. This process, however, does not account for how material thickness affects the appearance of the silicone. Presented herein is an innovative way to match the patient's skin tone using a gradated shade guide, such as a digitally designed gradated silicone carrier or guide, a device and methodology for use thereof that allows for the assessment of shade from the thin, marginal edge to approximately 5 mm thickness, as an example.

Therefore in accordance with various structural embodiments disclosed herein, a gradated shade guide for visual assessment of shade of a tinted material compared to a patient's skin tone at a plurality of depths of the tinted material is presented. The gradated shade carrier is operable to house for inspection varying thicknesses of the tinted material corresponding to the plurality of depths.

Further, in accordance with various method and process embodiments disclosed herein, a method of shade matching a tinted material to a patient's skin tone is presented. The method includes determining a thickness of the tinted material to achieve a color match of the tinted material compared to the patient's skin tone using a gradated shade guide that provides for visual assessment of shade of the tinted material, the gradated shade guide operable to house for inspection varying thicknesses of the tinted material corresponding to a plurality of depths and where said determining the thickness includes comparing to the patient's skin tone the tinted material housed at the plurality of depths in the gradated shade guide. Determining the thickness may include comparing to the patient's skin tone the tinted material housed in the gradated shade guide at the plurality of depths of the tinted material, the plurality of depths including a maximum depth and a marginal edge, to determine the thickness of the tinted material of the color match. Shade matching of the tinted material to the patient's skin tone may be initially determined by comparing the tinted material housed in a carrier tray of the gradated shade guide to the patient's skin as described herein. A prosthesis may be manufactured using the color match shade determined from judicious use of the gradated shade guide.

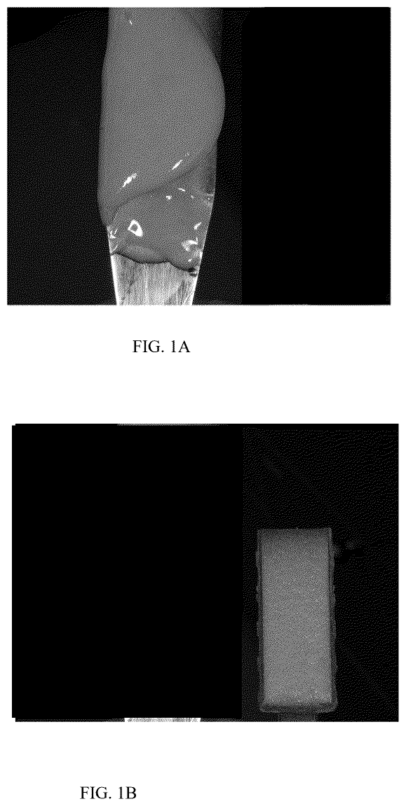

As noted, maxillofacial prosthetics is the branch of prosthodontics concerned with the restoration and replacement of stomatognathic and craniofacial structures with prostheses that may or may not be removed on a regular or elective basis. The prosthetic concealment of a congenital or acquired extraoral defect is challenging and heavily relies on the ability of the maxillofacial prosthodontist or anaplastologist to select the correct material, recreate the missing anatomy, and blend the correct tints to match the patient's skin tone. Material selection, generally comprised of poly(dimethylsiloxine) (PDMS) elastomers, is based upon the physical properties necessary to replace the missing anatomy. One important physical property of these materials is tear strength which provides resistance to permanent damage of the thin margins of the prosthesis, which is typically adhesively placed and removed daily. Although prosthetic materials have an abundance of research, advancements in anatomical re-creation are sparsely reported. The re-creation of the missing anatomy relies upon the artistic ability of the individual fabricating the prosthesis and without the appropriate tone selection, a prosthesis will never be well-concealed. Color matching may be completed by mixing pigments and rayon flocking into medical-grade silicone and using a flat carrier to assess the accuracy of the blend at only a given depth, shown in the traditional shade carrier of FIG. 1A. Color matching has always been a challenge to maxillofacial prosthodontists because the existing techniques have several inherent challenges: inconsistencies during tone production, variability of skin tone, and limited silicone-specific shade guides. Each challenge makes it difficult to accurately develop and assess the ideal shade. Several techniques have been proposed to mitigate these challenges such as producing prefabricated shade guides or utilizing a colorimeter for production of an intrinsic tone. These solutions do not address differences in material thickness and their effect on the appearance of the material.

As disclosed herein, an innovative method and structure to compare tinted silicone to the patient's skin tone using a digitally designed silicone carrier is presented. This scaled device allows for visual assessment of tinted silicone from a depth of approximately 5 mm, for example, to the thinnest margin. Reference to FIG. 1B illustrates a graded shade guide, in accordance with embodiments of the present disclosure.

A patient with a history of gross nasal resection due to stage 4 squamous cell carcinoma presented for fabrication of a nasal prosthesis. At the initial evaluation, the patient's chief complaint was the inability to clean the debris and mold out of her existing prosthesis. The patient also stated that previous maxillofacial prosthodontists have had difficulty matching her skin tone. During the clinical exam, it was noted that the body of the prosthesis blended well, but the margins were not well-blended. The marginal discrepancy ultimately failed to provide the concealment the patient desired.

In accordance with various embodiments herein, a gradated shade guide for visual assessment of shade of a tinted material compared to a patient's skin tone at a plurality of depths of the tinted material from a maximum depth to a marginal edge, the gradated shade carrier operable to house for inspection varying thicknesses of the tinted material from the maximum depth to the marginal edge of the plurality of depths is disclosed. As used herein, the gradated shade carrier, gradated shade guide and the like may be used interchangeably.

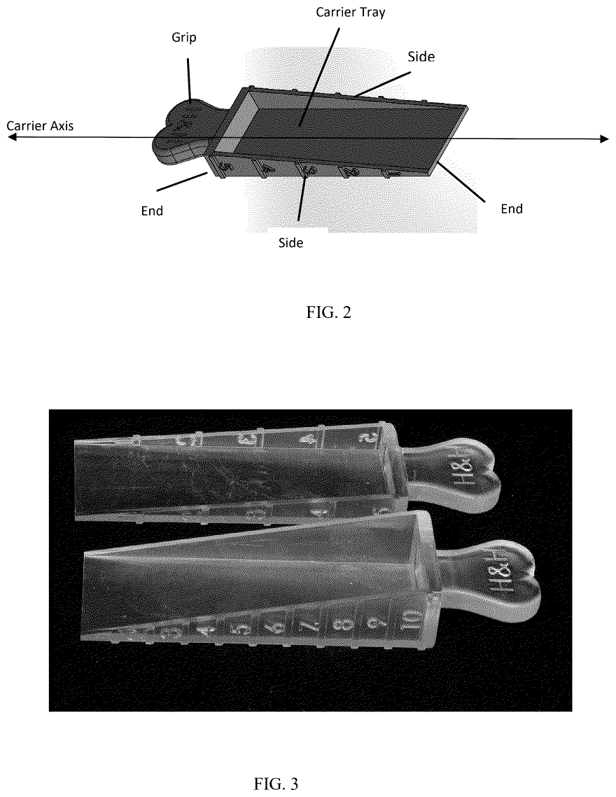

Utilizing parametric computer-aided design (CAD) soft-ware (SolidWorks; Dassault Système, Velizy-Villacoublay, France), a silicone carrier was designed, shown in FIG. 2, which illustrates a digital design of gradated silicone shade guide, in accordance with the present disclosure. The carrier was created to have a maximum height of approximately 5 mm on one end and decreased to approximately 0 over approximately 50 mm length over the carrier axis of the gradated shade guide, from a maximum depth at a first end of the carrier to a marginal edge at a second end of the carrier, as shown in FIG. 2. Gradations of 1-5 mm were placed on the external surface of the carrier and a finger grip or handle was placed on the 5 mm side for easier handling during shade assessment. This design was exported as a stereolithography (STL) file able to be staged in the software for additive manufacturing (3DSprint; 3D Systems, Rock Hill, South Carolina, USA). A prototype was printed (ProJet 6000 HD; 3D Systems, Rock Hill, South Carolina, USA) in a biocompatible Class 1 resin material (Accura ClearVue; 3D Systems, Rock Hill, South Carolina, USA) that resulted in a transparent product, shown in FIG. 3. Transparency of the gradated shade guide of course makes it easier for color matching as well as determining a color match, such as a best color match, of the tinted material compared to the patient's skin tone.

As can be seen in FIGS. 2 and 3, the gradated shade guide may be a carrier tray operable to house the tinted material and having a carrier axis from the maximum depth at a first end of the carrier tray to the marginal edge at a second end of the carrier tray, the first and second ends being opposing ends of the carrier tray. The gradated shade guide is scaled in depth from the maximum depth at the first end to the marginal edge at the second end of the carrier tray as indicated by gradations of the plurality of depths marked on the carrier tray.

FIG. 3 illustrates two configurations/variations of three-dimensional (3D) gradated silicon shade guides, in accordance with the present disclosure. It can be seen that the two example graded shade guides are wedges configured to house the tinted material of varying thicknesses.

The plurality of depths are indicated by gradations evenly scaled and marked along the carrier axis of the carrier tray of the gradated shade guide, which, in these examples, are marked along one or more external surfaces of the carrier tray. As noted herein, the gradations of the plurality of depths are marked along the carrier axis from approximately 50 mm at the maximum depth at the first end to approximately 0 mm at the marginal edge at the second end of the carrier tray, as shown in the top gradated shade guide of FIG. 3. Other maximum depths may be employed as well and as noted herein; the bottom gradated shade guide of FIG. 3 illustrates a maximum depth of 10 mm, for example. In both instances, the carrier tray is configured as a wedge or a step wedge in which opposing first and second sides each couple to first and second ends of the carrier tray. Gradations (5, 4, 3, 2, 1 mm in the case of the shade guide of FIG. 2 and the topmost shade guide of FIG. 3, or 10, 9, 8, 7, 6, 5, 4, 3, 2, 1 mm for the bottom shade guide of FIG. 3) of the plurality of depths are marked on one or more of the opposing first and second sides along the carrier axis from approximately 50 mm at the maximum depth at the first end to approximately 0 mm at the marginal edge at the second end of the carrier tray.

The tinted material housed in the gradated shade guide may be tinted silicone and the gradated shade guide is a gradated carrier operable to permit visual assessment of the tinted silicone compared to the patient's skin tone from the maximum depth to the marginal edge. The tinted silicon may be medical grade silicone including mixed pigments and rayon flocking.

Referring again to the wedge configuration of a gradated shade guide, the carrier tray is configured as a wedge and further includes opposing first and second sides that each couple to the first and second ends of the carrier tray and where gradations of the plurality of depths are marked along one or more of the opposing first and second sides from the maximum depth at the first end to the marginal edge at the second end, as indicated in FIGS. 2 and 3. Gradations of the plurality of depths are marked along one or more external surfaces of the opposing first and second sides. As shown in the embodiments of FIGS. 2 and 3, the opposing first and second sides of the carrier tray are approximately parallel along the carrier axis and the gradation markings along one or more of the opposing first and second sides are along the carrier axis, although other configurations could be employed within the spirit and scope of the disclosure.

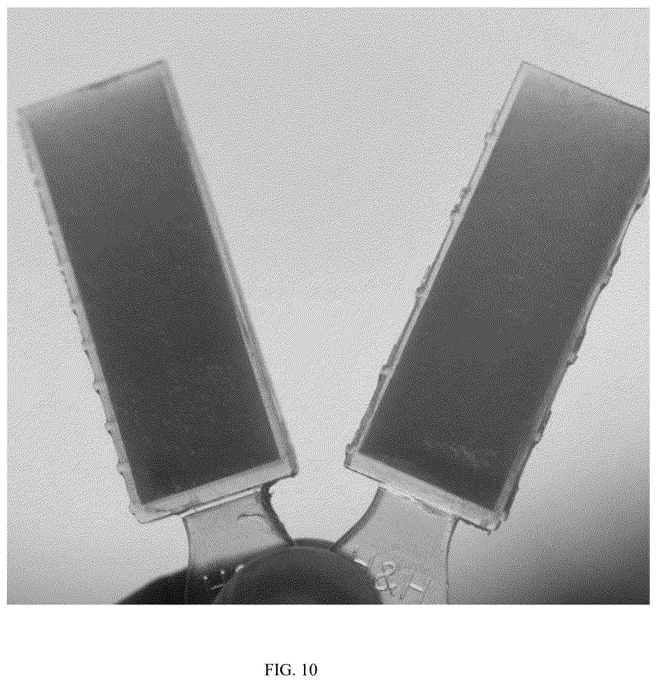

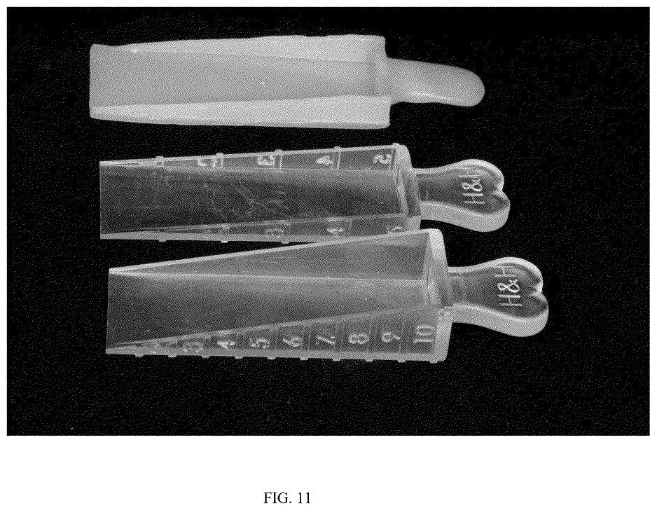

Further, as illustrated in FIGS. 2, 3, 9 and 10, the gradated shade guide may have a grip, such as a finger grip, or handle, coupled to the carrier tray. As shown in the drawings, the grip may be coupled to the first end of the carrier tray near the maximum depth of the carrier tray, although other locations and configurations of the grip are included herein. Various examples of gradated silicone shade guides with grips or handles and filled with medical grade silicone are illustrated in FIG. 10. Still further configurations of gradated silicone shade guides with grips or handles are illustrated in FIG. 11.

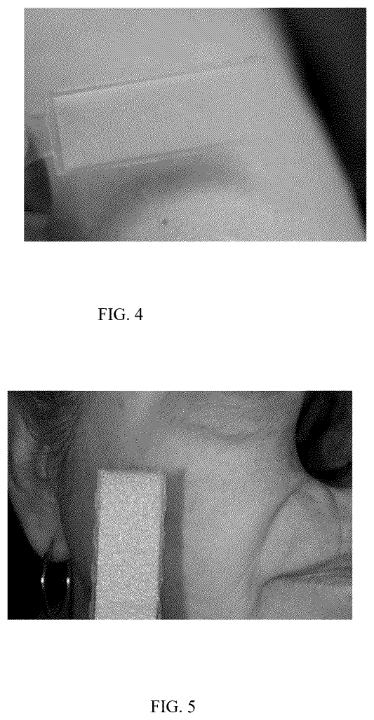

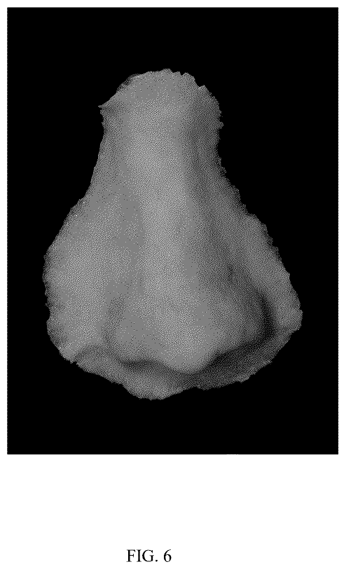

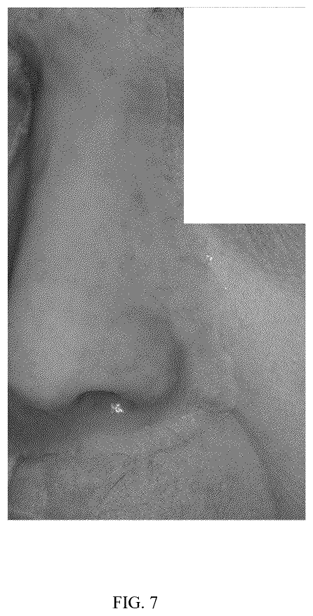

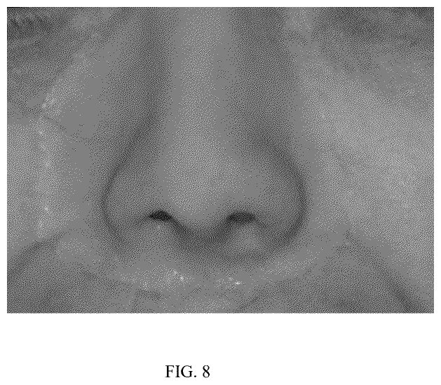

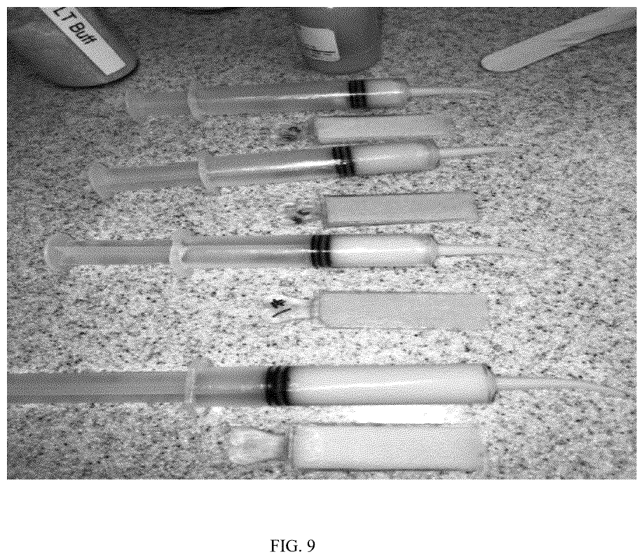

Silicone (A-588-1 RTV; Factor 2, Lakeside, Arizona, USA), silicone coloring pigment (Functional Intrinsic II—Silicone Coloring System; Factor 2, Lakeside, Arizona, USA), and rayon flocking (Factor 2, Lakeside, Arizona, USAZ) were used to create a base shade sample and three derivation samples to match the tissue around the patient's defect. Uncured samples were placed in the gradated silicone carrier to visually assess shade under natural lighting; FIG. 4 illustrates shade matching of unexposed skin. Once shade matching was completed, the optimal thickness was determined to achieve the best color match at the margin. This was performed for a variety of shades in order to achieve the best esthetic result; variations of silicone tones ready to be dispensed during prosthesis fabrication is shown in FIG. 5. These specific shades were selectively injected into the master cast and corresponding mold to create the final prosthesis. After polymerization, the prosthetic was tried on, further characterized with external tinting (Functional Extrinsic—Silicone Coloring System; Factor 2, Lakeside, Arizona, USA), sealed (TS-564; Factor 2, Lakeside, Arizona, USA), and finalized with matting dispersion (MD-564; Factor 2, Lakeside, Arizona, USA) to idealize the esthetics in a final prosthesis, as shown in FIG. 6. During delivery of the final prosthesis, such as that shown in FIG. 6, for example, the patient stated satisfaction with the result, shown in the final delivery of the final prosthesis to a patient in FIGS. 7 and 8. Gradated silicone shade guides with various colors of silicone, for example are shown in FIG. 9.

The goal of extraoral prosthesis fabrication is to provide the complete concealment of a facial defect while being comfortable for the patient and maintaining sufficient durability. Undertaking such a task is challenging, but can be achieved by using the correct material, re-creating the missing anatomy, and correctly determining the patient's skin tone.

Previous authors have explored numerous options to assist with determining an accurate tone. Over et al. described variable thicknesses of silicone and how well it matched a patient's skin tone using a colorimeter. It was determined that silicone thicknesses of 6, 8, and 10 mm had the highest correlation to that of the patient when utilizing a colorimeter for verification. Yet, in the same article the author stated there was a low correlation between patient skin colorimeter measurements and the silicone sample colorimeter measurement. As stated by Over et al., noted in the references listed below, one of the limitations of utilizing a colorimeter is that it is not known how deep a colorimeter penetrates human skin, especially since the article did not include patients with a variety of pigmentation. Due to the use of light with a colorimeter, an accurate measurement with transparent material may not be achievable until a substantial thickness of the material is present. Additionally, it is necessary to make a visually accurate sample before measuring with the colorimeter to color match. For these reasons, the use of a colorimeter seems appealing for replicating an existing prosthesis but is not practical or accurate for the initial fabrication.

A second version of improved gradated silicone carrier described herein, to a depth of approximately 10 mm, was fabricated for this patient but it was noted that the extra depth from approximately 5 mm to approximately 10 mm did not change the appearance of the sample when assessed clinically, and, therefore, the benefit may be attenuated for thicker material beyond approximately 5 mm. Moreover, given the cost of medical silicone, there is an incentive to not use more (thicker) when little or no improvement is seen.

The digitally designed gradated silicone carrier described herein provides an innovative tool (device) and way (method) to match the patient's skin tone and allows for assessment of shade from the thin marginal edge to approximately 5 mm of thickness. As described in the disclosure, an innovation includes a system relying on both the innovative device and method to compare tinted silicone to the patient's skin tone using the digitally designed silicone carrier. This scaled device allows for visual assessment of tinted silicone from a depth of approximately 5 mm to the thinnest margin. The gradated silicone shade guide dramatically improves the esthetic result. The method of use provides the means to visually compare a silicone sample to that of the patient from the thinnest margin to any desired thickness and allows for assessment of color based on thickness variability, leading to a more predictable outcome for extraoral prostheses, for example.

While the gradated silicone shade guide is discussed as having up to a depth of approximately 5 mm for the reasons set forth above, the innovation presented herein need not be so limited. The properties of silicone may differ and provide for the use of a shade guide that is deeper than approximately 5 mm without departing from the scope of the innovation described herein. FIG. 11, for example, illustrates one shade guide having a depth of approximately 5 mm while another shows a depth of approximately 10 mm. A shade guide may have another depths without departing from the scope described herein. Also, as illustrated in FIG. 11, the gradated shade guide need not clear or translucent; here, an example in pink is shown.

Color matching of maxillofacial prostheses is dependent upon the shade and thickness of the material. Utilization of the gradated silicone shade guide dramatically improves the esthetic result by allowing complete assessment of shades from the thin margin to variable depths of the prosthesis. The technique discussed herein provides the means to visually compare a silicone sample to that of the patient from the thinnest margin to any desired thickness. This device allows for assessment of color based on thickness variability, leading to a more predictable outcome for extraoral prostheses.

### REFERENCES

Glossary of Prosthodontic Terms (Ninth Edition). The glossary of prosthodontic terms. J Prosthet Dent. 2017;117:C1-e105. https://doi.org/10.1016/j.prosdent.2016.12.001.
Bell WT, Chalian VA, Moore BK. Polydimethyl siloxane materials in maxillofacial prosthetics: evaluation and comparison of physical properties. J Prosthet Dent. 1985;54(3):404-10.
Aziz T, Waters M, Jagger R. Analysis of the properties of silicone rubber maxillofacial prosthetic materials. J Dentistry. 2003;31(1):67-74. [Internet]. [cited 2023 Mar. 12] https://doi.org/10.1016/S0300-5712(02)00084-2.
Ranabhatt R, Singh K, Siddharth R, Tripathi S, Arya D. Color matching in facial prosthetics: a systematic review. J Indian Prosthodont Soc. 2017;17(1):3-7. [Internet]. [cited 2023 Mar. 12] https://doi.org/10.4103/0972-4052.197935.
Guttal S, Patil N, Nadiger R, Kulkarni R. A study on reproducing silicone shade guide for maxillofacial prostheses matching Indian skin color. Indian J Dent Res. 2008;19:191-95. 2008/19/3/191/42949 [serial online] [cited 2023 Mar. 12] https://www.ijdr.in/text.asp?
Anitha KV. A custom made intrinsic silicone shade guide for Indian population. J Clin Diagn Res. 2016;10(4):ZC27-30. [Internet]. [cited 2023 Mar. 12] https://doi.org/10.7860/JCDR/2016/17116.7539.
Over LM, Andres CJ, Moore BK, Goodacre CJ, Muñoz CA. Using a colorimeter to develop an intrinsic silicone shade guide for facial prostheses. J Prosthodont. 1998;7:237-49. [Internet] [cited 2023 Mar. 12] https://doi.org/10.1111/j.1532-849X.1998.tb00212.x.

The example and alternative embodiments described above may be combined in a variety of ways with each other without departing from the invention.

Embodiments of the disclosure have been described to explain the nature of the innovation. Those skilled in the art may make changes in the details, materials, steps and arrangement of the described embodiments within the principle and scope of the disclosure, as expressed in the appended claims.

While implementations of the disclosure are susceptible to embodiment in many different forms, there is shown in the drawings and will herein be described in detail specific embodiments, with the understanding that the present disclosure is to be considered as an example of the principles of the disclosure and not intended to limit the disclosure to the specific embodiments shown and described. In the description above, like reference numerals may be used to describe the same, similar or corresponding parts in the several views of the drawings.

In this document, relational terms such as first and second, top and bottom, and the like may be used solely to distinguish one entity or action from another entity or action without necessarily requiring or implying any actual such relationship or order between such entities or actions. The terms “comprises,” “comprising,” “includes,” “including,” “has,” “having,” or any other variations thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises . . . a” does not, without more constraints, preclude the existence of additional identical elements in the process, method, article, or apparatus that comprises the element.

Reference throughout this document to “one embodiment,” “certain embodiments,” “an embodiment,” “implementation(s),” “aspect(s),” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means “any of the following: A; B; C; A and B; A and C; B and C; A, B and C.” An exception to this definition will occur only when a combination of elements, functions, steps or acts are in some way inherently mutually exclusive. Also, grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth. References to items in the singular should be understood to include items in the plural, and vice versa, unless explicitly stated otherwise or clear from the text.

Recitation of ranges of values herein are not intended to be limiting, referring instead individually to any and all values falling within the range, unless otherwise indicated, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose. Ranges of values and/or numeric values are provided herein as examples only, and do not constitute a limitation on the scope of the described embodiments. The use of any and all examples, or exemplary language (“e.g.,” “such as,” “for example,” or the like) provided herein, is intended merely to better illuminate the embodiments and does not pose a limitation on the scope of the embodiments. No language in the specification should be construed as indicating any unclaimed element as essential to the practice of the embodiments.

For simplicity and clarity of illustration, reference numerals may be repeated among the figures to indicate corresponding or analogous elements. Numerous details are set forth to provide an understanding of the embodiments described herein. The embodiments may be practiced without these details. In other instances, well-known methods, procedures, and components have not been described in detail to avoid obscuring the embodiments described. The description is not to be considered as limited to the scope of the embodiments described herein.

In the following description, it is understood that terms such as “first,” “second,” “top,” “bottom,” “up,” “down,” “above,” “below,” and the like, are words of convenience and are not to be construed as limiting terms. Also, the terms apparatus, device, system, etc. may be used interchangeably in this text.

The many features and advantages of the disclosure are apparent from the detailed specification, and, thus, it is intended by the appended claims to cover all such features and advantages of the disclosure which fall within the scope of the disclosure. Further, since numerous modifications and variations will readily occur to those skilled in the art, it is not desired to limit the disclosure to the exact construction and operation illustrated and described, and, accordingly, all suitable modifications and equivalents may be resorted to that fall within the scope of the disclosure.

What is claimed is:

1. A gradated shade guide for visual assessment of shade of a tinted material compared to a patient's skin tone at a plurality of depths of the tinted material, the gradated shade carrier operable to house for inspection varying thicknesses of the tinted material corresponding to the plurality of depths.

2. The gradated shade guide of claim 1, where a maximum depth of the plurality of depths of the gradated shade guide is approximately 5 mm.

3. The gradated shade guide of claim 1, where the tinted material includes tinted silicone and the gradated shade guide is a gradated carrier operable to permit visual assessment of the tinted silicone compared to the patient's skin tone from a maximum depth of the plurality of depths to a marginal edge of the plurality of depths.

4. The gradated shade guide of claim 3, where the tinted silicone is medical grade silicone including mixed pigments and rayon flocking.

5. The gradated shade guide of claim 1, the gradated shade guide further comprising a carrier tray operable to house the tinted material and having a carrier axis from a maximum depth of the plurality of depths at a first end of the carrier tray to a marginal edge of the plurality of depths at a second end of the carrier tray, the first and second ends being opposing ends of the carrier tray.

6. The gradated shade guide of claim 5, where the gradated shade guide is scaled in depth from the maximum depth at the first end to the marginal edge at the second end of the carrier tray as indicated by gradations of the plurality of depths marked on the carrier tray.

7. The gradated shade guide of claim 6, where the plurality of depths are indicated by gradations evenly scaled and marked along the carrier axis of the carrier tray of the gradated shade guide.

8. The gradated shade guide of claim 7, where the plurality of depths are evenly scaled and gradations of the plurality of depths are marked along one or more external surfaces of the carrier tray.

9. The gradated shade guide of claim 7, where gradations of the plurality of depths are marked along the carrier axis having a length of approximately 50 mm from the maximum depth at the first end to the marginal edge at the second end of the carrier tray.

10. The gradated shade guide of claim 6, where the carrier tray is configured as a wedge and further includes opposing first and second sides that each couple to the first and second ends of the carrier tray and where gradations of the plurality of depths are marked along one or more of the opposing first and second sides from the maximum depth at the first end to the marginal edge at the second end.

11. The gradated shade guide of claim 10, where gradations of the plurality of depths are marked along one or more external surfaces of the opposing first and second sides.

12. The gradated shade guide of claim 10, where the opposing first and second sides of the carrier tray are approximately parallel along the carrier axis and the gradation markings along one or more of the opposing first and second sides are along the carrier axis.

13. The gradated shade guide of claim 5, the gradated shade guide further comprising a grip coupled to the carrier tray.

14. The gradated shade guide of claim 1, where the gradated shade guide is manufactured from one or more of a transparent material and a resin material.

15. A method of shade matching a tinted material to a patient's skin tone, comprising:
determining a thickness of the tinted material to achieve a color match of the tinted material compared to the patient's skin tone using a gradated shade guide that provides for visual assessment of shade of the tinted material, the gradated shade guide operable to house for inspection varying thicknesses of the tinted material corresponding to a plurality of depths and where said determining the thickness includes comparing to the patient's skin tone the tinted material housed at the plurality of depths in the gradated shade guide.

16. The method of claim 15, where said determining the thickness includes comparing to the patient's skin tone the tinted material housed in the gradated shade guide at the plurality of depths of the tinted material, the plurality of depths including a maximum depth and a marginal edge, to determine the thickness of the tinted material of the color match.

17. The method of claim 15, further comprising prior to determining the thickness of the tinted material determining shade matching of the tinted material to the patient's skin tone by comparing the tinted material housed in a carrier tray of the gradated shade guide to the patient's skin.

18. The method of claim 17, where said determining shade matching further comprises generating the tinted material by mixing one or more of pigments and flocking into medical grade silicone, housing the generated tinted material in the carrier tray of the gradated shade guide, and comparing the housed tinted material to the patient's skin tone.

19. The method of claim 15, further comprising manufacturing a prosthesis for the patient using the color match shade of the tinted material, including injecting the color match of the tinted material into a master cast from which the prosthesis is made.

20. The method of claim 19, said manufacturing the prosthesis further includes one or more of polymerizing tinted material of silicone in the master cast, externally tinting the prosthesis, and matting dispersion of the prosthesis.
